(19) United States
(12) Patent Application Publication
(10) Pub. No.: US 2026/0248582 A1
(43) Pub. Date: Aug. 27, 2026

(54) GRAFT PREPARATION DEVICE

(71) Applicant: EXPAND MEDICAL LTD., Amirim (IL)

(72) Inventors: Arie Levy, Shoam (IL); Ofek Levin, Amirim (IL); Lior Itzhaki, Shoham (IL)

(73) Assignee: Stryker European Operations Ltd., Carrigtwohill (IE)

(21) Appl. No.: 19/162,831

(22) PCT Filed: Mar. 1, 2024
(86) PCT No.: PCT/IB2024/000117

§ 371 Date: Sep. 5, 2025

Related U.S. Application Data
(60) Provisional application No. 63/450,056, filed on Mar. 5, 2023.

(51) Int. Cl. A61B 90/00

(52) U.S. Cl. CPC A61B 90/39; A61B 2090/395

(57) ABSTRACT

The present invention provides a method for preparing a surgical graft outside of the body. Specifically, the device is configured to hold a graft in place while a surgical tool, such as a scalpel, is used to puncture it. The device is comprised of a tray for the graft to sit in and a lid that features a plurality of guide holes that are large enough for a scalpel to completely pass through.

### TECHNICAL FIELD

The invention relates to devices that assist in preparing grafts for orthopedic surgery.

### BACKGROUND

Painful joint and tissue injuries may be caused by intense activity or age-related tissue deterioration. For example, ligament tears may arise from degenerative changes that occur with aging or from acute injuries during sports or other intensive activity. Treating those injuries to the musculoskeletal system is what defines the medical specialties of orthopedic surgery. Common orthopedic surgeries include repairs of the spine, shoulder, hand, hip, or knee, among others.

For example, some people may suffer from a tear to one of a group tendons in the shoulder known as the rotator cuff. A complete tear of a rotator cuff tendon can be very painful and cause the loss of arm function. Orthopedic surgeries address those injuries. Some approaches to orthopedic surgery use an implant, or graft, as a patch of biocompatible material such as collagen to anchor a separated tendon back to its original site of attachment.

Grafts show promise in orthopedic surgery but are not without drawbacks. For example, some arthroscopic surgery approaches require preparing a graft for delivery by pre-making various medial, posterior, anterior, and lateral cuts through the graft. See Forlizzi, 2021, Reconstruction with 6-mm acellular dermal allograft and knotless glenoid anchors, Arthroscopy Techniques 10(7):e1821-e1827, incorporated by reference. To perform that repair as described, the surgeon must cut twelve specific holes through the graft on a “back bench” while incisions are being made into the patient's shoulder for graft delivery. If there are any cutting errors, e.g., if the scalpel slices out to the edge of the graft for any hole, it is hoped that the error would be detected and the graft discarded so that a new graft could be prepared before insertion into the joint. If the holes are imperfect and undetected, the graft may not be fully secured in the joint and surgical recovery may be poor.

### SUMMARY

The invention provides devices and methods for preparing a graft for surgery. Devices of the invention provide a tray or platform on which to position a graft such as an acellular dermal allograft. The device includes guides, such as holes or slots or rails that position a tool such as a marker or a scalpel to thereby precisely control positioning of the tool with respect to the graft. A marker may be positioned using the guides and used to make marks on the graft and/or a knife or scalpel may be positioned using the guides and used to make cuts onto, into, or through the graft at precise positions. The device may be box-like, with a bottom portion constituting a tray or platform for holding a graft in position and a lid-portion that may be opened and closed with respect to the bottom portion. The lid may have a set of holes therethrough at positions corresponding to where cuts are to be made through a graft.

In use, a surgeon may place the device on a bench adjacent a surgical table. The surgeon may position a graft in the tray or platform portion of the device and close the lid. The surgeon may insert a tool through various holes of a set. For example, according to her preference, the surgeon may push a scalpel through the holes or a marker. The surgeon may prefer to first use the set of hole to mark the graft and, e.g., optionally to open the lid and confirm the positioning of the markings. After marking the graft, the surgeon may make incisions into the graft, optionally “freehand”, using the new markings as a visual guide, or by using the same guides provided by the device that were used for making the markings.

Because the guides (e.g., holes) in the device may be at locations corresponding to predetermined positions on the graft where it is desirable for incisions to be made in preparation for a surgery, the graft maybe reliably marked and cut, with accuracy and precision, to prepare the graft for surgery. Once a graft such as an acellular dermal allograft is marked and/or cut using devices and methods of the invention, the marked and cut graft may be delivered to a surgical site in a patient.

In certain aspects, the invention provides a method of preparing a graft for delivery in surgery. The method includes placing a surgical graft in a base of a graft preparation device; moving a top member of the graft preparation device into a closed position whereby the graft preparation device holds the graft in a fixed, pre-determined position within the graft preparation device; using a guide of the graft preparation device to bring a tool into contact with the graft at a specific position controlled by the guide; and marking or cutting the graft at the specific position with the tool. The guide may be a hole through the top member. The hole may be one of a set of holes at locations on the top member corresponding to predetermined positions on the graft for incisions to be made in preparation for a surgery such as rotator cuff repair. The tool may be a surgical marker and the method may include making marks on the graft. The method may include bringing a second tool with a blade (e.g., a scalpel) through the hole and making an incision into the graft with the second tool at the specific position.

The guide may be one of a set of holes, in which each hole has a substantially circular aperture extended by two opposed slots, such that the circular aperture guides a conical tip of surgical marker to mark the graft at the specific position and the two opposed slots guide a scalpel to pierce the graft at the specific position.

The guide may be one of a set of holes at locations on the top member corresponding to predetermined positions on the graft for incisions to be made in preparation for a surgery, and the method may include inserting a surgical marker through one or more of the holes to thereby mark the graft at the predetermined positions; making incisions into the graft at one or more of the predetermined positions; and delivering the marked and cut graft to a surgical site in a patient.

In certain embodiments, the top member of the graft preparation device is connected to the base via a hinge, and moving the top member into the closed position includes fastening the top member to the base via a releasable latch. The method may include removing the prepared graft from the graft preparation device by grasping an edge of the graft through a cutaway in a wall of the base and lifting the graft out of the graft preparation device.

In related aspects, the invention provides a device for preparing a surgical graft. The device includes a base with one or more raised walls dimensioned to hold a surgical graft in a predetermined position with respect to the device; a top member movable between an open position and a closed position with respect to the base; and a guide such as a hole positioned on the top member. When a graft is in the predetermined position and when the top member is in the closed position, the guide guides a surgical tool to make contact with the graft at a specific position on the graft controlled by the guide. The guide may be one of a set of holes through the top member, at locations on the top member corresponding to predetermined positions on the graft for incisions to be made in preparation for a surgery. Each hole of the device may have a substantially circular aperture extended by two opposed slots, wherein the circular aperture guides a conical tip of surgical marker to mark the graft at the specific position and wherein the two opposed slots guide a scalpel to pierce the graft at the specific position. The device may have multiple sets of holes, e.g., a first set of holes through the top member, wherein the first set of holes correspond to predetermined positions for incisions to be made in a graft for preparation for a first surgery, and a second set of holes useful for preparing a graft for a second surgery (e.g., for different sized grafts). Optionally the first set of holes are all labeled with a first mark, and the second set of holes are all labeled with a second mark.

The top member of the graft preparation device may be connected to the base via a hinge. The device may include a releasable latch for fastening the top member to the base. The top member may include a protrusion, such as a leaf, that holds the graft is in the predetermined position by pressure when the top member is in the closed position. The device may include a cutaway in a wall of the base wherein a surgeon can grasp a graft that is in the device. The top member may include a tab to aid in opening and closing the top member. The base may include a notch to accommodate the tab when the top member is in the closed position. The base may include receiving one or more hole(s) in alignment with the guide(s) when the top member in the closed position. The device may include a locking mechanism such as a snap latch configured to hold the top member to the base. The base may include an outline or recess on a floor of the base to aid in placement of a small graft. The top member may include a deformable leaf that exerts pressure on the graft when the graft is in the predetermined position and when the top member is in the closed position to prevent motion of the graft relative to the device. The deformable leaf may comprise a three-side cut out of the lid, with a central portion sloped downward into the interior volume of the base when the lid is in the closed position. Preferably, the base is deep enough to accommodate the graft and the deformable leaf with the top member in the closed position. The guide may be one hole of a set of holes, wherein one or more holes of the set of holes are large enough for a blade of a surgical scalpel to pass completely through.

Aspects of the present invention provide a device for use in puncturing a graft for orthopedic surgery. The device is primarily useful for grafts that are difficult to puncture during a surgical procedure, like those that are thicker or made from a tough material. The device comprises a tray with at least two walls and a floor defining an interior volume configured to accommodate a graft and a lid that overlays the tray. The lid contains a plurality of holes wherein each is large enough for a surgical scalpel to pass completely through. The device allows a surgeon to take a graft that would otherwise be difficult to puncture during surgery and prepare punctures ahead of time and outside of the body. The guide holes and holding leaf ensure the punctures will be accurately placed. To further reduce potential mistakes, a marker can be used with the device in place of a scalpel to mark where on the graft the punctures will be made. This gives surgeons the opportunity to double check if the placement of the puncture site is correct, potentially saving a patient from having to deal with an improperly punctured graft and the negative effects it can have on their recovery, and saving the hospital the expense of starting over with a new graft.

In some embodiments, the lid contains a protrusion, such as a tab, to assist in opening and closing the lid. The tray may include at least one notch for the tab to rest on when the lid is in the closed position. In a preferred embodiment, there are two notches that sit opposite each other that hold the tab on either side of it. The notches also effectively act as a “stop” to prevent a user from closing the lid too much and crushing the graft inside. Between these two notches, the wall of the device may have a portion that is cutaway or concave, allowing for easy removal of the graft.

In another embodiment, the tray includes a protrusion from the floor that is used to stabilize the device and assist in opening it. In a preferred embodiment, there are two protrusions on either side of the concave portion of the wall, sometimes referred to as base tabs.

In a preferred embodiment, the lid contains at least one hole for which a surgical scalpel can pass completely through. In a preferred embodiment there are a plurality of holes to allow a user to puncture a graft in multiple different places. The tray contains holes, also known as receiving holes, that are aligned with those on the lid when the lid is in the closed position, allowing a scalpel to pass through the graft and into those holes. The size of the puncture hole is determined by the depth with which the scalpel is pushed through. If the scalpel is pushed further through the holes, then the size of the perforations in the graft will be larger.

In a certain embodiment, the tray includes features which assist in graft placement. In a preferred embodiment, there is a raised portion, such as a raised step, that is used for grafts that are the same or a similar size as the interior of the tray. In a preferred embodiment there are outlines or recess on the floor of the interior of the tray which serve as guide lines for placing grafts that are smaller.

In some embodiments, the lid contains a three-side cut out that slopes downward into the interior volume of the tray when the lid is in the closed position. This protrusion, also known as a holding leaf, secures the graph in place when the lid is in the closed position. In a preferred embodiment, the holding leaf is made of a flexible material so that the graft is not crushed. Preferably, the tray is deep enough to accommodate a graft and the holding leaf while the lid is in the closed position.

In another embodiment, the device includes a locking mechanism that keeps the lid closed against the tray while it is being used. In a preferred embodiment, the locking mechanism is a snap latch.

### BRIEF DESCRIPTION OF THE DRAWINGS

FIG. 1 shows a device for puncturing grafts.

FIG. 2 shows the device with a graft placed inside.

FIG. 3 shows the device with the lid closed.

FIG. 4 shows a surgical scalpel being pushed through the holes on the lid of the device.

FIG. 5 shows a marking tool being pushed through the holes on the lid of the device.

FIG. 6 shows a graft with marks and punctures after being used in the device.

### DETAILED DESCRIPTION

The present invention provides a device to assist in puncturing surgical grafts. The device may include a base or bottom portion or tray configured to hold a graft and a lid or top member overlaying the tray containing guides or holes for a tool to pass through.

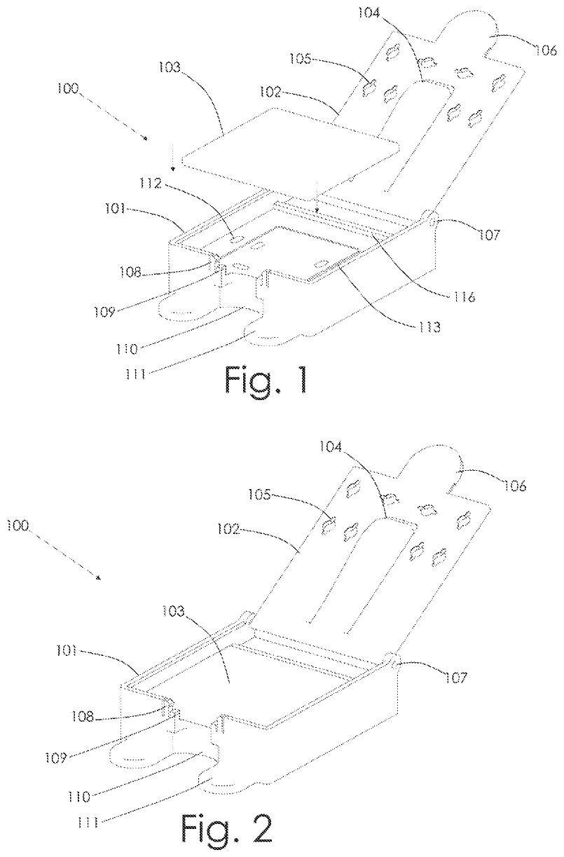

FIG. 1 shows a graft preparation device 100. The device 100 is useful for preparing a surgical graft 103. The device 100 includes a base 101 with one or more raised walls 116 dimensioned to hold a surgical graft 103 in a predetermined position with respect to the device 100. A top member 102 is movable between an open position and a closed position with respect to the base 101. The device 100 includes a guide 105 (such as a hole) positioned on the top member 102. The guide 105 may be a hole through the top member. In certain embodiments, the guide 105 is one of a set of holes through the top member, wherein the set of holes are at locations on the top member 102 corresponding to predetermined positions on the graft 103 for incisions to be made in preparation for a surgery.

Various features may be included for the device 100. In some embodiments, the top member 102 features a flexible protrusion 104 that acts e.g., as a holding leaf to keep the graft in place during use. The lid preferably includes at least one guide 105 which may be, for example, one of a plurality, or “set” of holes. A device may have multiple separate “sets” of holes. The device 100 may include of a first set of holes through the top member, wherein the first set of holes correspond to predetermined positions for incisions to be made in a graft 103 for preparation for a first surgery, wherein the top member 102 comprises a second set of holes useful for preparing a graft 103 for a second surgery, wherein the first set of holes are all labeled with a first mark, and the second set of holes are all labeled with a second mark.

In a preferred embodiment, the holes are large enough to let a surgical scalpel 114 pass completely through. The lid also features a protrusion that acts as a tab 106 to help open and close the device 100. The top member 102 of the graft 103 preparation device may be connected to the base 101 via a hinge 107. The device may include a releasable latch 108 for fastening the top member 102 to the base.

To stabilize the graft 103 and ensure accurate use, the device 100 may be able to lock shut. In a preferred embodiment, the locking mechanism is a snap latch 108. Next to and just below the snap latch 108 is a notch 109 that the tab 106 rests on when the lid is in the closed position. In a preferred embodiment, the notch 109 also acts as a “stop” to prevent the top member 102 from crushing the graft 103. In the present embodiment, the notch 109 and snap latch 108 are carved out from the wall of the base 101.

To assist with graft 103 removal, the device 100 may include a cutaway 110 in a wall of the base 101 wherein a surgeon can grasp a graft 103 that is in the device. The cutaway 110 may define a concave portion. The cutaway 110 may allow the graft 103 to hang slightly over the wall, making it easier to remove. In a preferred embodiment, the cutaway 110 is located between the snap latches 108 and notches 109.

The base 101 has a base tab 111 extending from the floor that aids in stabilizing the base 101 and opening the top member 102. In a preferred embodiment, there are two base tabs 111. In another embodiment, the base tabs 111 are parallel to each other and located on either side of the concave portion 110 of the wall.

The base 101 may include receiving holes 112 that are aligned with the guides 105, e.g., holes on the top member 102 when the top member 102 is in the closed position. The receiving holes 112 allow the surgical scalpel 114 to fully puncture through the graft 103.

To assist in placing the graft, the wall 116 may include a raised step inside the base 101. This raised step 116 assists in placing grafts that are the same or a similar size as the tray. If there is a graft 103 that is smaller than the base 101, then a recess or guide lines 113 may be provided on a floor of the base 101 to assist with placement.

FIG. 2 is a perspective view of the device 100 with the graft 103 placed inside the base 101. In a preferred embodiment, the graft 103 sits between the raised step 116 and the front wall of the base 101. The graft 103 also hangs slightly over the concave portion 110, allowing a user to remove it with ease. In another preferred embodiment, the graft 103 may be smaller than the tray and will not rest against the raised step 116. In this case, the graft 103 may be placed within or on the guide lines 113 located on the floor of the base 101. In this embodiment, the graft 103 is still hanging slightly over the concave portion 110 of the base 101.

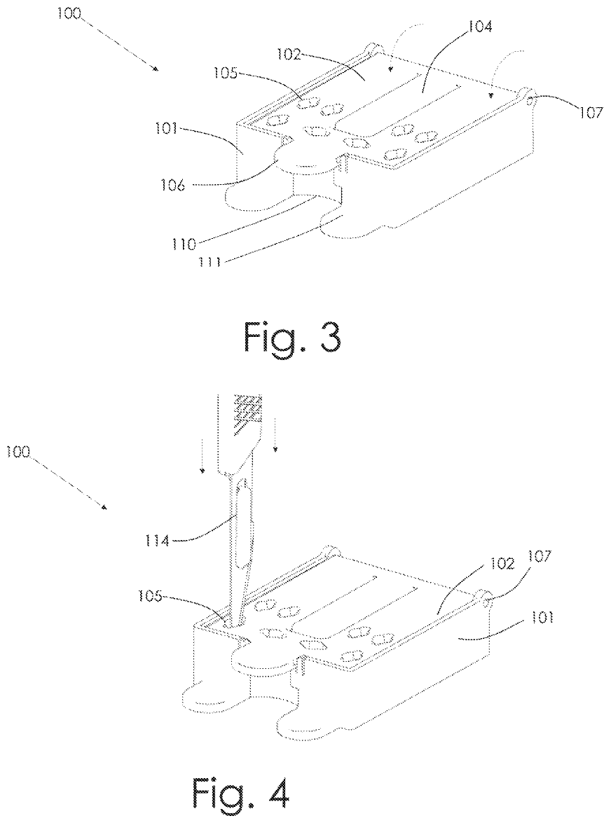

FIG. 3 shows the device 100 with the top member 102 in the closed position. When a graft 103 is in the predetermined position and when the top member 102 is in the closed position, guide 105 a surgical tool (e.g., scalpel 114 or marker 115) to make contact with the graft 103 at a specific position on the graft 103 controlled by the guide. Here, the tab 106 overhangs the concave portion 110 and is resting on the notches 109. The three-side cut out of the holding leaf is visible even when the top member 102 is closed. In a preferred embodiment, the base 101 is deep enough to accommodate a graft 103 with the top member 102 in the closed position.

The base 101 (bottom portion or tray) may itself include receiving holes 112 aligned with guides 105 such as holes when the lid or top member is in the closed position. The guides and/or receiving holes 112 allow the surgical tool to pierce completely through the graft. The base or tray may also have feature for facilitating the placement of a graft. For example, the base or tray may have a raised step in the interior that the graft can be placed against. Placing the graft flush against the raised step ensures the graft is placed correctly within the device. The base or tray may include protrusions or tabs extending out from the floor of the device. The protrusions or tabs may help stabilize the device and assist in opening the lid or top member. The lid also has a protrusion extending out from it. This protrusion acts as a tab for a user to use when moving the lid into an open and closed position. As a safety, the tray comprises a notch for the tab of the lid to rest on when it is closed. The notch prevents the lid from being closed too far and potentially crushing the graft inside. In a preferred embodiment there are two notches opposite each other on the wall of the tray. The wall between these notches is concave, curving in towards the interior of the tray. When the graft is placed, the graft will hang slightly over the concave portion, allowing for easy removal from the tray.

The device 100 may include a locking system such as a snap latch with, e.g., a pair of latches. The snap latches may be on either side of a cutaway 110 or concave portion of the wall and behind notches used to hold the top member 102 or lid.

FIG. 4 shows the device in a closed position for use preparing a graft, e.g., for an orthopedic surgery such as a rotator cuff repair. A surgical scalpel 114 is being pushed into one of the guides 105, e.g., holes on the top member 102 to penetrate the graft 103 inside the base 101. The size of the perforation can be controlled by how deep the scalpel 114 is pushed through the guide hole 105. Preferably, the base 101 is deep enough to accommodate the graft 103 and the deformable leaf with the top member 102 in the closed position. When the guide 105 is one hole of a set of holes, one or more of the holes may be large enough for a blade of a surgical scalpel to pass completely through. The deeper the scalpel 114 goes, the larger the perforation.

In some embodiments, the guide 105 is one hole of a set of holes, wherein each hole has a substantially circular aperture extended by two opposed slots, wherein the circular aperture guides a conical tip of surgical marker to mark the graft 103 at the specific position and wherein the two opposed slots guide 105 a scalpel to pierce the graft 103 at the specific position.

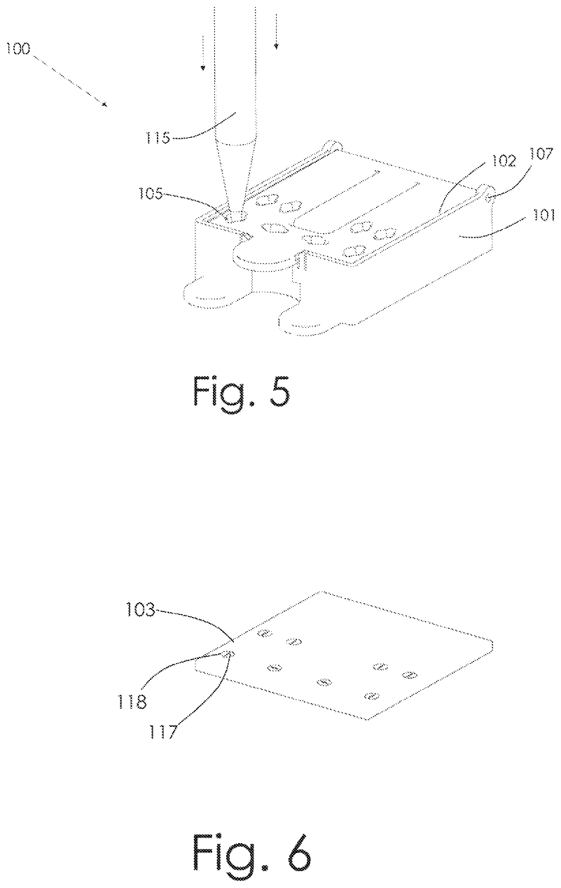

FIG. 5 illustrates the device in use in a method of preparing a graft 103 for delivery in surgery such as an orthopedic surgery (e.g., rotator cuff repair). The method includes placing a surgical graft 103 in a base 101 of a graft 103 preparation device; moving a top member 102 of the graft 103 preparation device into a closed position whereby the graft 103 preparation device holds the graft 103 in a fixed, pre-determined position within the graft 103 preparation device; using a guide 105 (e.g., hole through the top member) of the graft preparation device to bring a tool into contact with the graft 103 at a specific position controlled by the guide; and marking or cutting the graft 103 at the specific position with the tool. Here, a marker 115 is used in the guide holes on the top member 102 to mark the graft 103 that is in the base 101. The marker 115 can be any tool that would be suitable for use in leaving a mark on the graft 103 such as a sterile surgical marking pen.

As shown, the guide 105 is one hole of a set of holes at locations on the top member 102 corresponding to predetermined positions on the graft 103 for incisions to be made in preparation for a surgery. The method may include bringing a second tool comprising a blade (i.e., a scalpel) through the hole and making an incision into the graft 103 with the second tool at the specific position.

When the guide 105 is a hole through the top member, the hole may have a substantially circular aperture extended by two opposed slots (see FIG. 1). The circular aperture may guide a conical tip of surgical marker 115 (see FIG. 5) to mark the graft 103 at the specific position. The two opposed slots may guide a scalpel 114 (see FIG. 4) to pierce the graft 103 at the specific position(s).

Methods of the invention may include inserting a surgical marker 115 through each hole of the set of holes to thereby mark the graft 103 at the predetermined positions and making incisions into the graft 103 at one or more of the predetermined positions.

FIG. 6 illustrates a graft 103 that has been used in the device 100. The graft 103 has been marked with a marker 115, leaving behind marks 118 to show where the perforations 117 will be made. The graft 103 also has perforations 117. In a preferred embodiment, the device 100 can be used with a marker 115 first to visualize where the perforations 117 will go on the graft. This allows for subsequent adjustment if necessary. The guides 105 may be at locations corresponding to predetermined positions on the graft 103 for incisions to be made in preparation for a surgery (see FIG. 6).

The method may include removing the prepared graft 103 from the graft 103 preparation device by grasping an edge of the graft 103 through a cutaway in a wall of the base 101 and lifting the graft 103 out of the graft 103 preparation device. The method preferably includes delivering the marked and cut graft 103 to a surgical site in a patient.

1-29. (canceled)

30. A method of preparing a patch formed of biocompatible material for delivery to a surgical site at a rotator cuff of a patient, the method comprising:
associating the patch formed of biocompatible material with a flexible portion of a graft preparation device positioned adjacent to a surgical table; and
marking and/or cutting the patch formed of biocompatible material at at least one position on the patch.

31. The method of claim 30, wherein the graft preparation device includes at least one hole, wherein the marking and/or cutting the patch formed of biocompatible material at the at least one position includes using the at least one hole to bring a tool into contact with the patch formed of biocompatible material at the at least one position.

32. The method of claim 31, wherein the at least one hole is one of at least two holes corresponding to positions on the patch formed of biocompatible material, wherein the marking and/or cutting includes forming an incision into the patch formed of biocompatible material using the tool at the at least one position determined by at least one of the at least two holes.

33. The method of claim 30, wherein the patch formed of biocompatible material includes collagen.

34. The method of claim 30, wherein the marking and/or cutting of the patch formed of biocompatible material includes marking the patch formed of biocompatible material using a surgical marker.

35. The method of claim 34, wherein marking and/or cutting the patch formed of biocompatible material further comprises passing a blade through the patch formed of biocompatible material and past the flexible portion of the graft preparation device.

36. The method of claim 35, wherein the marking of the patch formed of biocompatible material using the surgical marker and the passing the blade through the patch formed of biocompatible material occur at the same at least one position on the patch.

37. The method of claim 30, further comprising delivering the patch formed of biocompatible material to the surgical site at the rotator cuff of the patient.

38. The method of claim 30, wherein the marking and/or cutting the patch formed of biocompatible material includes passing a blade through the patch formed of biocompatible material and past the flexible portion of the graft preparation device.

39. The method of claim 30, wherein the marking and/or cutting the patch formed of biocompatible material includes penetrating the patch at the at least one position.

40. The method of claim 30, wherein the marking and/or cutting the patch formed of biocompatible material includes forming an incision into the patch at the at least one position.

41. The method of claim 30, wherein the graft preparation device includes a cutaway in a wall of the graft preparation device, the method further comprising, after the marking and/or cutting, removing the patch formed of biocompatible material from the graft preparation device by grasping a portion of the patch formed of biocompatible material via the cutaway.

42. The method of claim 30, wherein the graft preparation device includes a positioning aid, wherein the associating the patch formed of biocompatible material with the flexible portion of the graft preparation device includes using the positioning aid to associate the patch with the graft preparation device.

43. A device for preparing a patch formed of biocompatible material, the device comprising a structure including a flexible portion configured to contact the patch formed of biocompatible material, wherein the structure is configured to, with the patch formed of biocompatible material in contact with the flexible portion of the structure, allow a surgical tool to make contact with the patch formed of biocompatible material at at least one position on the patch formed of biocompatible material.

44. The device of claim 43, wherein the structure further includes at least one hole configured to guide a tool into contact with the patch formed of biocompatible material at the at least one position.

45. The device of claim 44, wherein the at least one hole is a substantially circular aperture extended by two opposed slots, wherein the substantially circular aperture is configured to guide a conical tip of a surgical marker to mark the patch formed of biocompatible material at the at least one position, and wherein the two opposed slots are configured to guide a scalpel to pierce the patch formed of biocompatible material at the at least one position.

46. The device of claim 43, further comprising a cutaway in a wall of the structure configured to allow a surgeon to grasp the patch formed of biocompatible material in contact with the device.

47. The device of claim 43, wherein the structure includes a positioning aid configured to aid in placement of the patch formed of biocompatible material into contact with the structure.

48. The device of claim 43, wherein the flexible portion includes a deformable leaf configured to exert pressure on the patch formed of biocompatible material when the patch formed of biocompatible material is in contact with the flexible portion to prevent motion of the patch formed of biocompatible material relative to the device.

49. The device of claim 43, wherein the device is configured to be positioned adjacent a surgical table to prepare the patch formed of biocompatible material to be delivered into a surgical site in a patient.
